(19) United States
(12) Patent Application Publication
(10) Pub. No.: US 2003/0228676 A1
(43) Pub. Date: Dec. 11, 2003

(54) Aggrecanase molecules

(76) Inventors: Michael J. Agostino, Andover, MA (US); Elizabeth DiBlasio, Tyngsboro, MA (US); Edward R. Lavallie, Harvard, MA (US); Bethany A. Freeman, Arlington, MA (US); Lisa A. Racie, Acton, MA (US); Weilan Zeng, Waltham, MA (US)

Correspondence Address: WYETH, PATENT LAW GROUP, FIVE GIRALDA FARMS, MADISON, NJ, 07940, US

(21) Appl. No.: 10/330,176

(22) Filed: Dec. 30, 2002

Related U.S. Application Data
(60) Provisional application No. 60/344,895, filed on Dec. 31, 2001.

(51) Int. Cl. C12N 9/64; C07H 21/04; C12P 21/02; C12N 5/06

(52) U.S. Cl. 435/226; 435/69.1; 435/348; 435/320.1; 536/23.2

(57) ABSTRACT

Novel aggrecanase proteins and the nucleotide sequences encoding them as well as processes for producing them are disclosed. Methods for developing inhibitors of the aggrecanase enzymes and antibodies to the enzymes for treatment of conditions characterized by the degradation of aggrecan are also disclosed.

### RELATED APPLICATION

This application relies on the benefit of priority of U.S. provisional patent application No. 60/344,895, filed on Dec. 31, 2001.

### FIELD OF THE INVENTION

The present invention relates to the discovery of nucleotide sequences encoding novel aggrecanase molecules, the aggrecanase proteins and processes for producing them. The invention further relates to the development of inhibitors of and antibodies to the aggrecanase enzymes. These inhibitors and antibodies may be useful for the treatment of various aggrecanase-associated conditions including osteoarthritis.

### BACKGROUND OF THE INVENTION

Aggrecan is a major extracellular component of articular cartilage. It is a proteoglycan responsible for providing cartilage with its mechanical properties of compressibility and elasticity. The loss of aggrecan has been implicated in the degradation of articular cartilage in arthritic diseases. Osteoarthritis is a debilitating disease which affects at least 30 million Americans (MacLean et al., J Rheumatol 25:2213-8 (1998)). Osteoarthritis can severely reduce quality of life due to degradation of articular cartilage and the resulting chronic pain. An early and important characteristic of the osteoarthritic process is loss of aggrecan from the extracellular matrix (Brandt and Mankin, Pathogenesis of Osteoarthritis, in Textbook of Rheumatology, WB Saunders Company, Philadelphia, Pa., at 1355-1373 (1993)). The large, sugar-containing portion of aggrecan is thereby lost from the extra-cellular matrix, resulting in deficiencies in the biomechanical characteristics of the cartilage.

A proteolytic activity termed “aggrecanase” is thought to be responsible for the cleavage of aggrecan thereby having a role in cartilage degradation associated with osteoarthritis and inflammatory joint disease. Work has been conducted to identify the enzyme responsible for the degradation of aggrecan in human osteoarthritic cartilage. Two enzymatic cleavage sites have been identified within the interglobular domain of aggrecan. One (Asn³⁴¹-Phe³⁴²) is observed to be cleaved by several known metalloproteases. Flannery et al., J Biol Chem 267:1008-14 (1992); Fosang et al., Biochemical J. 304:347-351 (1994). The aggrecan fragment found in human synovial fluid, and generated by IL-1 induced cartilage aggrecan cleavage is at the Glu³⁷³-Ala³⁷⁴ bond (Sandy et al., J Clin Invest 69:1512-1516 (1992); Lohmander et al., Arthritis Rheum 36: 1214-1222 (1993); Sandy et al., J Biol Chem 266: 8683-8685 (1991)), indicating that none of the known enzymes are responsible for aggrecan cleavage in vivo.

Recently, identification of two enzymes, aggrecanase-1 (ADAMTS 4) and aggrecanase-2 (ADAMTS-11) within the “Disintegrin-like and Metalloprotease with Thrombospondin type 1 motif” (ADAM-TS) family have been identified which are synthesized by IL-1 stimulated cartilage and cleave aggrecan at the appropriate site (Tortorella et al., Science 284:1664-6 (1999); Abbaszade et al., J Biol Chem 274: 23443-23450 (1999)). It is possible that these enzymes could be synthesized by osteoarthritic human articular cartilage. It is also contemplated that there are other, related enzymes in the ADAM-TS family which are capable of cleaving aggrecan at the Glu³⁷³-Ala³⁷⁴ bond and could contribute to aggrecan cleavage in osteoarthritis. There is a need to identify other aggrecanase enzymes and determine ways to block their activity.

### SUMMARY OF THE INVENTION

The present invention is directed to the identification of novel aggrecanase protein molecules capable of cleaving aggrecan, the nucleotide sequences which encode the aggrecanase enzymes, and processes for the production of aggrecanases. These enzymes are contemplated to be characterized as having proteolytic aggrecanase activity. The invention further includes compositions comprising these enzymes.

The invention also includes antibodies to these enzymes, in one embodiment, for example, antibodies that block aggrecanase activity. In addition, the invention includes methods for developing inhibitors of aggrecanase which block the enzyme's proteolytic activity. These inhibitors and antibodies may be used in various assays and therapies for treatment of conditions characterized by the degradation of articular cartilage.

The invention provides an isolated DNA molecule comprising a DNA sequence chosen from: the sequence of SEQ ID NO. 1 from nucleotide #1-#3675; fragments of SEQ ID NO. 1, sequences which hybridize under stringent conditions with SEQ ID NO. 1, naturally occurring human allelic sequences, and equivalent degenerative codon sequences.

The invention also comprises a purified aggrecanase protein comprising an amino acid sequence chosen from: the amino acid sequence of SEQ ID NO. 2 from amino acid #1-#1224; fragments of SEQ ID NO. 2, and homologous aggrecanase proteins comprising addition, substitution, and deletion mutants of the sequences.

The invention also provides a method for producing a purified aggrecanase protein produced by the steps of culturing a host cell transformed with a DNA molecule according to the invention, and recovering and purifying from said culture medium a protein comprising the amino acid sequence set forth in SEQ ID NO. 2.

The invention also provides an antibody that binds to a purified aggrecanase protein of the invention. It also provides a method for developing inhibitors of aggrecanase comprising the use of aggrecanase protein chosen from SEQ ID NO. 2, and a fragment thereof.

Additionally, it provides a pharmaceutical composition for inhibiting the proteolytic activity of aggrecanase, wherein the composition comprises at least one antibody according to the invention and at least one pharmaceutical carrier. It also provides a method for inhibiting aggrecanase in a mammal comprising administering to said mammal an effective amount of the pharmaceutical composition and allowing the composition to inhibit aggrecanase activity.

### BRIEF DESCRIPTION OF THE FIGURES AND SEQUENCES

The following table provides information on each of the sequences provided in the application.

FIG. 1 is the full length nucleotide sequence of ADAMTS16 (EST17).

FIG. 2 is the predicted amino acid sequence for ADAMTS16.

| SEQUENCES | FIGURES | DESCRIPTION |
|---|---|---|
| 1 | | full length nucleotide |
| | | sequence of ADAMTS16 |
| | | (EST17) |
| 2 | | predicted a.a. seq. of |
| | | ADAMTS16 |
| 3 | | zinc binding signature |
| | | region of aggrecanase-1 |
| 4 | | primer |
| 5 | | primer |
| 6 | | primer |
| 7 | | primer |
| 8 | | primer |
| 9 | | primer |
| 10 | | primer |
| 11 | | primer |
| 12 | | synthesized nucleotides |
| 13 | | synthesized nucleotides |
| 14 | | synthesized nucleotides |
| 15 | | synthesized nucleotides |
| 16 | | nucleotide insert |
| 17 | | nucleotide sequence |
| | | containing an Xhol insert |
| 18 | | a 68 base pair adapter |
| | | nucleotide sequence |

### DETAILED DESCRIPTION OF THE INVENTION

### I. Novel Aggrecanase Molecules

In one embodiment, the nucleotide sequence of an aggrecanase molecule of the present invention is set forth in SEQ ID NO. 1, as nucleotides #1 to #3675. The invention further includes equivalent degenerative codon sequences of the sequence set forth in SEQ ID NO. 1, as well as fragments thereof which exhibit aggrecanase activity. The full length sequence of the aggrecanase of the present invention may be obtained using the sequences of SEQ ID NO. 1 to design probes for screening for the full sequence using standard techniques.

The amino acid sequence of the isolated aggrecanase-like molecule is set forth in SEQ ID. NO. 2, as amino acid #1 to #1224. See S. Cal et al., “Cloning, Expression Analysis, and Structural Characterization of Seven Human ADAMTSs, a Family of Metalloproteinases with Disintegrin and Thrombospondin-1 Domains,” Gene, 283:49-62 (2002). The proposed leader sequence is contemplated to comprise amino acids #1-#24. The proposed pro domain is contemplated to comprise amino acids #25-#279 (probable PACE-Furin processing site RHKR is underlined). The proposed metalloprotease domain is contemplated to comprise amino acids #280-#497 with catalytic Zn binding domain located at amino acids #430-#444, and Met turn located at amino acid #458. The proposed disintegrin domain is contemplated to comprise amino acids #498-#586. The proposed thrombospondin type I domain is contemplated to comprise amino acids #587-#662. The proposed cysteine rich and cysteine poor spacer domain is contemplated to comprise amino acids #663-#873. The proposed thrombospondin type I submotifs (5) are contemplated to comprise amino acids #874-#1180 (amino acids #874-#927; amino acids #931-#986; amino acids #990-#1047; amino acids #1055-#1114; and amino acids #1130-#1180). The proposed PLAC domain is contemplated to comprise amino acids #1181-#1224. The proposed RGD site comprises amino acids #71-73. N-linked glycosylation sites comprise amino acids #156-#158, #310-#312, #741-#743, #780-#782, #835-#837, #905-#907, and #935-#937.

The invention further includes fragments of the amino acid sequence which encode molecules exhibiting aggrecanase activity.

The invention includes methods for obtaining the full length aggrecanase molecule, the DNA sequence obtained by this method and the protein encoded thereby. The method for isolation of the full length sequence involves utilizing the aggrecanase sequence set forth in SEQ ID NO. 1 to design probes for screening, or otherwise screen, using standard procedures known to those skilled in the art. The preferred sequence for designing probes is SEQ ID NO. 1.

The human aggrecanase protein or a fragment thereof may be produced by culturing a cell transformed with a DNA sequence chosen from SEQ ID NO. 1 and recovering and purifying from the culture medium a protein characterized by an amino acid sequence set forth in SEQ ID NO. 2 substantially free from other proteinaceous materials with which it is co-produced. For production in mammalian cells, the DNA sequence further comprises a DNA sequence encoding a suitable propeptide 5′ to and linked in frame to the nucleotide sequence encoding the aggrecanase enzyme.

The human aggrecanase proteins produced by the method discussed above are characterized by having the ability to cleave aggrecan and having an amino acid sequence chosen from SEQ ID NO. 2, variants of the amino acid sequence of SEQ ID NO. 2, including naturally occurring allelic variants, and other variants in which the proteins retain the ability to cleave aggrecan characteristic of aggrecanase proteins. Preferred proteins include a protein which is at least about 80% homologous, and more preferably at least about 90% homologous, to the amino acid sequence shown in SEQ ID NO. 2. Finally, allelic or other variations of the sequences of SEQ ID NO. 2 whether such amino acid changes are induced by mutagenesis, chemical alteration, or by alteration of DNA sequence used to produce the protein, where the peptide sequence still has aggrecanase activity, are also included in the present invention. The present invention also includes fragments of the amino acid sequence of SEQ ID NO. 2 which retain the activity of aggrecanase protein.

### II. Identification of Homologous Aggrecanase Proteins and DNA Encoding Them

It is expected that additional human sequences and other species have DNA sequences homologous to human aggrecanase enzymes. The invention, therefore, includes methods for obtaining the DNA sequences encoding other aggrecanase proteins, the DNA sequences obtained by those methods, and the protein encoded by those DNA sequences. This method entails utilizing the nucleotide sequence of the invention or portions thereof to design probes to screen libraries for the corresponding gene from other species or coding sequences or fragments thereof from using standard techniques. Thus, the present invention may include DNA sequences from other species, which are homologous to the human aggrecanase protein and can be obtained using the human sequence. The present invention may also include functional fragments of the aggrecanase protein, and DNA sequences encoding such functional fragments, as well as functional fragments of other related proteins. The ability of such a fragment to function is determinable by assay of the protein in the biological assays described for the assay of the aggrecanase protein.

For example, SEQ ID NO. 1 was used as a query against GenBank and GeneSeq to find homologous human sequences. Several sequences were identified as similar to SEQ ID NO. 1, i.e., that is various clones of the same gene. These sequences were identified by the following accession numbers: Ax319852 (sequence 16 from WO 01/83783), AJ315734, AB095949, AX481380, AC010269, AC022424, AC091978, AC091967, Abk49822, Abk49826, Abk49825, Aal43654, Abk86137, Abk49821, Abk49827, Abs59326, Abs59323, Abs59325, Abk90322, Abs59324, Aas85554, Aas70328, Aas70239.

Moreover, SEQ ID NO. 1 was used to search a protein database (BLASTX), which combines records from several databases. This search revealed several nonhuman homologous sequences. The published sequences include the following accession numbers: NCBI:25053113, NCBI:AAH34739_{—}1, NCBI:2088361, GENESEQ:AAB50004 (bovine), NCBI:CAA65253_{—}1, NCBI:21288693 (bovine), GENESEQ:AAW47030, GENESEQ:AAY53898, NCBI:20898418, NCBI:CAA93287_{—}1, NCBI:AAD34012_{—}1 (rat), NCBI:AAG29823_{—}1 (rat), NCBI:BAA24501_{—}1 (mouse), SWISSPROT:ATS1_MOUSEP, NCBI:BAA11088_{—}1, GENESEQ:AAY53900 (bovine), NCBI:AAF46065_{—}2 (fruitfly), GENESEQ:ABB60410 (fruitfly), GENESEQ:AAY53899 (mouse), NCBI:AAN17331_{—}1, NCBI:20861058, NCBI:25056874, GENESEQ:AAU79499 (mouse), GENESEQ:AAU80151 (mouse), NCBI:AAM50192_{—}1 (fruitfly), NCBI:AAF55199_{—}2 (fruitfly), NCBI:23634336, NCBI:AAD56356_{—}1 (mouse), GENESEQ:AAB72280 (mouse), and GENESEQ:AAB21252 (rat). It is expected that these sequences, from non-human species, are homologous to human aggrecanase enzymes.

BLASTX was also used to search protein databases with ADAMTS16 (SEQ ID NO. 1) to find sequences with extensive enough differences to suggest alternative splicing. The DNA sequences encoding these splicing candidates were compared to SEQ ID NO. 1. Some of the sequences found during the search include the following accession numbers: NCBI:AJ315734, GeneSeq:ABK86137, GeneSeq:AB59323, GeneSeq:ABS59324, GeneSeq:ABS59325, and GeneSeq:ABS59326.

Several ESTs are also published in GenBank, including the following accession numbers: BM808410, BM906555, BM850160, BM845044, BM845406, BM844919, and BF933693.

The aggrecanase molecules provided herein also include factors encoded by sequences similar to those of SEQ ID NO. 1, but into which modifications or deletions are naturally provided (e.g. allelic variations in the nucleotide sequence which may result in amino acid changes in the protein) or deliberately engineered. For example, synthetic proteins may wholly or partially duplicate continuous sequences of the amino acid residues of SEQ ID NO. 2. These sequences, by virtue of sharing primary, secondary, or tertiary structural and conformational characteristics with aggrecanase proteins may possess biological properties in common therewith. It is known, for example that numerous conservative amino acid substitutions are possible without significantly modifying the structure and conformation of a protein, thus maintaining the biological properties as well. For example, it is recognized that conservative amino acid substitutions may be made among amino acids with basic side chains, such as lysine (Lys or K), arginine (Arg or R) and histidine (His or H); amino acids with acidic side chains, such as aspartic acid (Asp or D) and glutamic acid (Glu or E); amino acids with uncharged polar side chains, such as asparagine (Asn or N), glutamine (Gln or Q), serine (Ser or S), threonine (Thr or T), and tyrosine (Tyr or Y); and amino acids with nonpolar side chains, such as alanine (Ala or A), glycine (Gly or G), valine (Val or V), leucine (Leu or L), isoleucine (Ile or I), proline (Pro or P), phenylalanine (Phe or F), methionine (Met or M), tryptophan (Trp or W) and cysteine (Cys or C). Thus, these modifications and deletions of the native aggrecanase may be employed as biologically active substitutes for naturally-occurring aggrecanase and in the development of inhibitors or other proteins in therapeutic processes. It can be readily determined whether a given variant of aggrecanase maintains the biological activity of aggrecanase by subjecting both aggrecanase and the variant of aggrecanase, as well as inhibitors thereof, to the assays described in the examples.

Other specific mutations of the sequences of aggrecanase proteins described herein involve modifications of glycosylation sites. These modifications may involve O-linked or N-linked glycosylation sites. For instance, the absence of glycosylation or only partial glycosylation results from amino acid substitution or deletion at asparagine-linked glycosylation recognition sites. The asparagine-linked glycosylation recognition sites comprise tripeptide sequences which are specifically recognized by appropriate cellular glycosylation enzymes. These tripeptide sequences are either asparagine-X-threonine or asparagine-X-serine, where X is usually any amino acid. A variety of amino acid substitutions or deletions at one or both of the first or third amino acid positions of a glycosylation recognition site (and/or amino acid deletion at the second position) results in non-glycosylation at the modified tripeptide sequence. Additionally, bacterial expression of aggrecanase-related protein will also result in production of a non-glycosylated protein, even if the glycosylation sites are left unmodified.

### III. Novel Aggrecanase Nucleotide Sequences

Still a further aspect of the invention are DNA sequences coding for expression of an aggrecanase protein having aggrecanase proteolytic activity or other disclosed activities of aggrecanase. Such sequences include the sequence of nucleotides in a 5′ to 3′ direction illustrated in SEQ ID NO. 1 and DNA sequences which, but for the degeneracy of the genetic code, are identical to the DNA sequence of SEQ ID NO. 1 and encode an aggrecanase protein.

Further included in the present invention are DNA sequences which hybridize under stringent conditions with the DNA sequence of SEQ ID NO. 1 and encode a protein having the ability to cleave aggrecan. Preferred DNA sequences include those which hybridize under stringent conditions (see Maniatis et al, Molecular Cloning (A Laboratory Manual), Cold Spring Harbor Laboratory, at 387-389 (1982)). Such stringent conditions comprise, for example, 0.1× SSC, 0.1% SDS, at 65° C. It is generally preferred that such DNA sequences encode a protein which is at least about 80% homologous, and more preferably at least about 90% homologous, to the sequence set forth in SEQ ID NO. 2. Finally, allelic or other variations of the sequences of SEQ ID NO. 1 whether such nucleotide changes result in changes in the peptide sequence or not, but where the peptide sequence still has aggrecanase activity, are also included in the present invention. The present invention also includes fragments of the DNA sequence shown in SEQ ID NO. 1 which encodes a protein which retains the activity of aggrecanase.

Similarly, DNA sequences which code for aggrecanase proteins coded for by the sequence of SEQ ID NO. 1 or aggrecanase proteins which comprise the amino acid sequence of SEQ ID NO. 2 but which differ in codon sequence due to the degeneracies of the genetic code or allelic variations (naturally-occurring base changes in the species population which may or may not result in an amino acid change) also encode the novel factors described herein. Variations in the DNA sequence of SEQ ID NO. 1 which are caused by point mutations or by induced modifications (including insertion, deletion, and substitution) to enhance the activity, half-life or production of the proteins encoded are also encompassed in the invention.

The DNA sequences of the present invention are useful, for example, as probes for the detection of mRNA encoding aggrecanase in a given cell population. Thus, the present invention includes methods of detecting or diagnosing genetic disorders involving the aggrecanase, or disorders involving cellular, organ or tissue disorders in which aggrecanase is irregularly transcribed or expressed. Antisense DNA sequences may also be useful for preparing vectors for gene therapy applications. Antisense DNA sequences are also useful for in vivo methods, such as to introduce the antisense DNA into the cell, to study the interaction of the antisense DNA with the native sequences, and to test the capacity of a promoter operatively linked to the antisense DNA in a vector by studying the interaction of antisense DNA in the cell as a measure of how much antisense DNA was produced.

A further aspect of the invention includes vectors comprising a DNA sequence as described above in operative association with an expression control sequence therefor. These vectors may be employed in a novel process for producing an aggrecanase protein of the invention in which a cell line transformed with a DNA sequence encoding an aggrecanase protein in operative association with an expression control sequence therefor, is cultured in a suitable culture medium and an aggrecanase protein is recovered and purified therefrom. This process may employ a number of known cells both prokaryotic and eukaryotic as host cells for expression of the protein. The vectors may be used in gene therapy applications. In such use, the vectors may be transfected into the cells of a patient ex vivo, and the cells may be reintroduced into a patient. Alternatively, the vectors may be introduced into a patient in vivo through targeted transfection.

### IV. Production of Aggrecanase Proteins

Another aspect of the present invention provides a method for producing novel aggrecanase proteins. The method of the present invention involves culturing a suitable cell line, which has been transformed with a DNA sequence encoding an aggrecanase protein of the invention, under the control of known regulatory sequences. The transformed host cells are cultured and the aggrecanase proteins recovered and purified from the culture medium. The purified expressed proteins are substantially free from other proteins with which they are co-produced as well as from other contaminants. The recovered purified protein is contemplated to exhibit proteolytic aggrecanase activity cleaving aggrecan. Thus, the proteins of the invention may be further characterized by the ability to demonstrate aggrecanase proteolytic activity in an assay which determines the presence of an aggrecan-degrading molecule. These assays or the development thereof is within the knowledge of one skilled in the art. Such assays may involve contacting an aggrecan substrate with the aggrecanase molecule and monitoring the production of aggrecan fragments (see for example, Hughes et al., Biochem J 305: 799-804 (1995); Mercuri et al, J Bio Chem 274:32387-32395 (1999)).

Suitable cells or cell lines may be mammalian cells, such as Chinese hamster ovary cells (CHO). The selection of suitable mammalian host cells and methods for transformation, culture, amplification, screening, product production and purification are known in the art. (See, e.g., Gething and Sambrook, Nature, 293:620-625 (1981); Kaufman et al, Mol Cell Biol, 5(7):1750-1759 (1985); Howley et al, U.S. Pat. No. 4,419,446.) Another suitable mammalian cell line, which is described in the accompanying examples, is the monkey COS-1 cell line. The mammalian cell CV-1 may also be suitable.

Bacterial cells may also be suitable hosts. For example, the various strains of E. coli (e.g., HB101, MC1061) are well-known as host cells in the field of biotechnology. Various strains of B. subtilis, Pseudomonas, other bacilli and the like may also be employed in this method. For expression of the protein in bacterial cells, DNA encoding the propeptide of aggrecanase is generally not necessary.

Many strains of yeast cells known to those skilled in the art may also be available as host cells for expression of the proteins of the present invention. Additionally, where desired, insect cells may be utilized as host cells in the method of the present invention. See, e.g., Miller et al., Genetic Engineering, 8:277-298 (Plenum Press 1986).

Another aspect of the present invention provides vectors for use in the method of expression of these novel aggrecanase proteins. Preferably the vectors contain the full novel DNA sequences described above which encode the novel factors of the invention. Additionally, the vectors contain appropriate expression control sequences permitting expression of the aggrecanase protein sequences. Alternatively, vectors incorporating modified sequences as described above are also embodiments of the present invention. Additionally, the sequence of SEQ ID NO. 1 or other sequences encoding aggrecanase proteins could be manipulated to express composite aggrecanase proteins. Thus, the present invention includes chimeric DNA molecules encoding an aggrecanase protein comprising a fragment from SEQ ID NO. 2 linked in correct reading frame to a DNA sequence encoding another aggrecanase protein.

The vectors may be employed in the method of transforming cell lines and contain selected regulatory sequences in operative association with the DNA coding sequences of the invention which are capable of directing the replication and expression thereof in selected host cells. Regulatory sequences for such vectors are known to those skilled in the art and may be selected depending upon the host cells. Such selection is routine and does not form part of the present invention.

### V. Generation of Antibodies

The purified proteins of the present inventions may be used to generate antibodies, either monoclonal or polyclonal, to aggrecanase and/or other aggrecanase-related proteins, using methods that are known in the art of antibody production. Thus, the present invention also includes antibodies to aggrecanase or other related proteins. The antibodies include both those that block aggrecanase activity and those that do not. The antibodies may be useful for detection and/or purification of aggrecanase or related proteins, or for inhibiting or preventing the effects of aggrecanase. The aggrecanase of the invention or portions thereof may be utilized to prepare antibodies that specifically bind to aggrecanase.

The term “antibody” as used herein, refers to an immunoglobulin or a part thereof, and encompasses any protein comprising an antigen binding site regardless of the source, method of production, and characteristics. The term includes but is not limited to polyclonal, monoclonal, monospecific, polyspecific, non-specific, humanized, single-chain, chimeric, synthetic, recombinant, hybrid, mutated, DCR-grafted antibodies. It also includes, unless otherwise stated, antibody fragments such as Fab, F(ab′)₂, Fv, scFv, Fd, dAb, and other antibody fragments which retain the antigen binding function.

Antibodies can be made, for example, via traditional hybridoma techniques (Kohler and Milstein, Nature 256:495-499 (1975)), recombinant DNA methods (U.S. Pat. No. 4,816,567), or phage display techniques using antibody libraries (Clackson et al., Nature 352: 624-628 (1991); Marks et al, J. Mol Biol. 222:581-597 (1991)). For various other antibody production techniques, see Antibodies: A Laboratory Manual, eds. Harlow et al., Cold Spring Harbor Laboratory (1988).

An antibody “specifically” binds to at least one novel aggrecanase molecule of the present invention when the antibody will not show any significant binding to molecules other than at least one novel aggrecanase molecule. The term is also applicable where, e.g., an antigen binding domain is specific for a particular epitope, which is carried by a number of antigens, in which case the specific binding member (the antibody) carrying the antigen binding domain will be able to bind to the various antigens carrying the epitope. In this fashion it is possible that an antibody of the invention will bind to multiple novel aggrecanase proteins. Typically, the binding is considered specific when the affinity constant Kₐ is higher than 10⁸ M⁻¹. An antibody is said to “specifically bind” or “specifically react” to an antigen if, under appropriately selected conditions, such binding is not substantially inhibited, while at the same time non-specific binding is inhibited. Such conditions are well known in the art, and a skilled artisan using routine techniques can select appropriate conditions. The conditions are usually defined in terms of concentration of antibodies, ionic strength of the solution, temperature, time allowed for binding, concentration of non-related molecules (e.g., serum albumin, milk casein), etc.

Proteins are known to have certain biochemical properties including sections which are hydrophobic and sections which are hydrophilic. The hydrophobic sections would most likely be located in the interior of the structure of the protein while the hydrophilic sections would most likely be located in the exterior of the structure of the protein. It is believed that the hydrophilic regions of a protein would then correspond to antigenic regions on the protein. The hydrophobicity of SEQ ID NO. 2 was determined using GCG PepPlot. The results indicated that the N-terminus is hydrophilic thereby reflecting the nature of the secretion signal peptide. Also, no transmembrane proteins were predicted.

### VI. Development of Inhibitors

Various conditions such as osteoarthritis are known to be characterized by degradation of aggrecan. Therefore, an aggrecanase protein of the present invention which cleaves aggrecan may be useful for the development of inhibitors of aggrecanase. The invention therefore provides compositions comprising an aggrecanase inhibitor. The inhibitors may be developed using the aggrecanase in screening assays involving a mixture of aggrecan substrate with the inhibitor followed by exposure to aggrecan. Inhibitors can be screened using high throughput processes, such as by screening a library of inhibitors. Inhibitors can also be made using three-dimensional structural analysis and/or computer aided drug design. The compositions may be used in the treatment of osteoarthritis and other conditions exhibiting degradation of aggrecan.

The method may entail the determination of binding sites based on the three dimensional structure of aggrecanase and aggrecan and developing a molecule reactive with the binding site. Candidate molecules are assayed for inhibitory activity. Additional standard methods for developing inhibitors of the aggrecanase molecule are known to those skilled in the art. Assays for the inhibitors involve contacting a mixture of aggrecan and the inhibitor with an aggrecanase molecule followed by measurement of the aggrecanase inhibition, for instance by detection and measurement of aggrecan fragments produced by cleavage at an aggrecanase susceptible site. Inhibitors may be proteins or small molecules.

### VII. Administration

Another aspect of the invention therefore provides pharmaceutical compositions containing a therapeutically effective amount of aggrecanase antibodies and/or inhibitors, in a pharmaceutically acceptable vehicle. Aggrecanase-mediated degradation of aggrecan in cartilage has been implicated in osteoarthritis and other inflammatory diseases. Therefore, these compositions of the invention may be used in the treatment of diseases characterized by the degradation of aggrecan and/or an up regulation of aggrecanase. The compositions may be used in the treatment of these conditions or in the prevention thereof.

The invention includes methods for treating patients suffering from conditions characterized by a degradation of aggrecan or preventing such conditions. These methods, according to the invention, entail administering to a patient needing such treatment, an effective amount of a composition comprising an aggrecanase antibody or inhibitor which inhibits the proteolytic activity of aggrecanase enzymes.

The antibodies and inhibitors of the present invention are useful to prevent, diagnose, or treat various medical disorders in humans or animals. In one embodiment, the antibodies can be used to inhibit or reduce one or more activities associated with the aggrecanase protein, relative to an aggrecanase protein not bound by the same antibody. Most preferably, the antibodies and inhibitors inhibit or reduce one or more of the activities of aggrecanase relative to the aggrecanase that is not bound by an antibody. In certain embodiments, the activity of aggrecanase, when bound by one or more of the presently disclosed antibodies, is inhibited at least 50%, may be inhibited at least 60, 62, 64, 66, 68, 70, 72, 72, 76, 78, 80, 82, 84, 86, or 88%, more preferably at least 90, 91, 92, 93, or 94%, and even more preferably at least 95% to 100% relative to an aggrecanase protein that is not bound by one or more of the presently disclosed antibodies.

Generally, the compositions are administered so that antibodies/their binding fragments are given at a dose from 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg to 1 mg/kg, and 500 μg/kg to 1 mg/kg. Preferably, the antibodies are given as a bolus dose, to maximize the circulating levels of antibodies for the greatest length of time after the dose. Continuous infusion may also be used after the bolus dose.

In another embodiment and for administration of inhibitors, such as proteins and small molecules, an effective amount of the inhibitor is a dosage which is useful to reduce the activity of aggrecanase to achieve a desired biological outcome. Generally, appropriate therapeutic dosages for administering an inhibitor may range from 5 mg to 100 mg, from 15 mg to 85 mg, from 30 mg to 70 mg, or from 40 mg to 60 mg. Inhibitors can be administered in one dose, or at intervals such as once daily, once weekly, and once monthly. Dosage schedules can be adjusted depending on the affinity for the inhibitor to the aggrecanase target, the half-life of the inhibitor, and the severity of the patient's condition. Generally, inhibitors are administered as a bolus dose, to maximize the circulating levels of inhibitor. Continuous infusions may also be used after the bolus dose.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Antibodies and inhibitors, which exhibit large therapeutic indices, are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any antibody and inhibitor used in the present invention, the therapeutically effective dose can be estimated initially from cell culture assays. A dose may be formulated in animal models to achieve a circulating plasma concentration range that includes the IC₅₀ (i.e., the concentration of the test antibody which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription-based assays, GDF protein/receptor binding assays, creatine kinase assays, assays based on the differentiation of pre-adipocytes, assays based on glucose uptake in adipocytes, and immunological assays.

The therapeutic methods of the invention include administering the aggrecanase inhibitor compositions topically, systemically, or locally as an implant or device. The dosage regimen will be determined by the attending physician considering various factors which modify the action of the aggrecanase protein, the site of pathology, the severity of disease, the patient's age, sex, and diet, the severity of any inflammation, time of administration and other clinical factors. Generally, systemic or injectable administration will be initiated at a dose which is minimally effective, and the dose will be increased over a preselected time course until a positive effect is observed. Subsequently, incremental increases in dosage will be made limiting such incremental increases to such levels that produce a corresponding increase in effect, while taking into account any adverse affects that may appear. The addition of other known factors, to the final composition, may also affect the dosage.

Progress can be monitored by periodic assessment of disease progression. The progress can be monitored, for example, by x-rays, MRI or other imaging modalities, synovial fluid analysis, patient perception, and/or clinical examination.

### VIII. Assays and Methods of Detection

The inhibitors and antibodies of the invention can be used in assays and methods of detection to determine the presence or absence of, or quantify aggrecanase in a sample. The inhibitors and antibodies of the present invention may be used to detect aggrecanase proteins, in vivo or in vitro. By correlating the presence or level of these proteins with a medical condition, one of skill in the art can diagnose the associated medical condition or determine its severity. The medical conditions that may be diagnosed by the presently disclosed inhibitors and antibodies are set forth above.

Such detection methods for use with antibodies are well known in the art and include ELISA, radioimmunoassay, immunoblot, western blot, immunofluorescence, immuno-precipitation, and other comparable techniques. The antibodies may further be provided in a diagnostic kit that incorporates one or more of these techniques to detect a protein (e.g., an aggrecanase protein). Such a kit may contain other components, packaging, instructions, or other material to aid the detection of the protein and use of the kit. When protein inhibitors are used in such assays, protein-protein interaction assays can be used.

Where the antibodies and inhibitors are intended for diagnostic purposes, it may be desirable to modify them, for example, with a ligand group (such as biotin) or a detectable marker group (such as a fluorescent group, a radioisotope or an enzyme). If desired, the antibodies (whether polyclonal or monoclonal) may be labeled using conventional techniques. Suitable labels include fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. Other suitable binding partners include biotin and avidin or streptavidin, IgG and protein A, and the numerous receptor-ligand couples known in the art.

### EXAMPLES

### Example 1

### Isolation of DNA

Potential novel aggrecanase family members were identified using a database screening approach. Aggrecanase-1 (Science 284:1664-1666 (1999)) has at least six domains: signal, propeptide, catalytic domain, disintegrin, tsp and c-terminal. The catalytic domain contains a zinc binding signature region, TAAHELGHVKF (SEQ. ID NO. 3) and a “MET turn” which are responsible for protease activity. Substitutions within the zinc binding region in the number of the positions still allow protease activity, but the histidine (H) and glutamic acid (E) residues must be present. The thrombospondin domain of Aggrecanase-1 is also a critical domain for substrate recognition and cleavage. It is these two domains that determine our classification of a novel aggrecanase family member. The coding region of the aggrecanase-1 DNA sequence was used to query against the GeneBank ESTs focusing on human ESTs using TBLASTN. The resulting sequences were the starting point in the effort to identify a full length sequence for potential family members. The nucleotide sequence of the aggrecanase of the present invention is ADAMTS16, also known as EST17.

ADAMTS16, GenBank accession No. BF933693, predicts a peptide having similarity to a portion of the TSP1 and Cysteine Rich Spacer domains of ADAMTS4. ADAMTS16 was located on the human genome (Celera Discovery System and Celera's associated databases) (Rockville, Md., USA) and a precomputed gene prediction (Genscan) was used to extend ADAMTS16 sequence. This predicted sequence was used to design primers to isolate the full length gene for ADAMTS16.

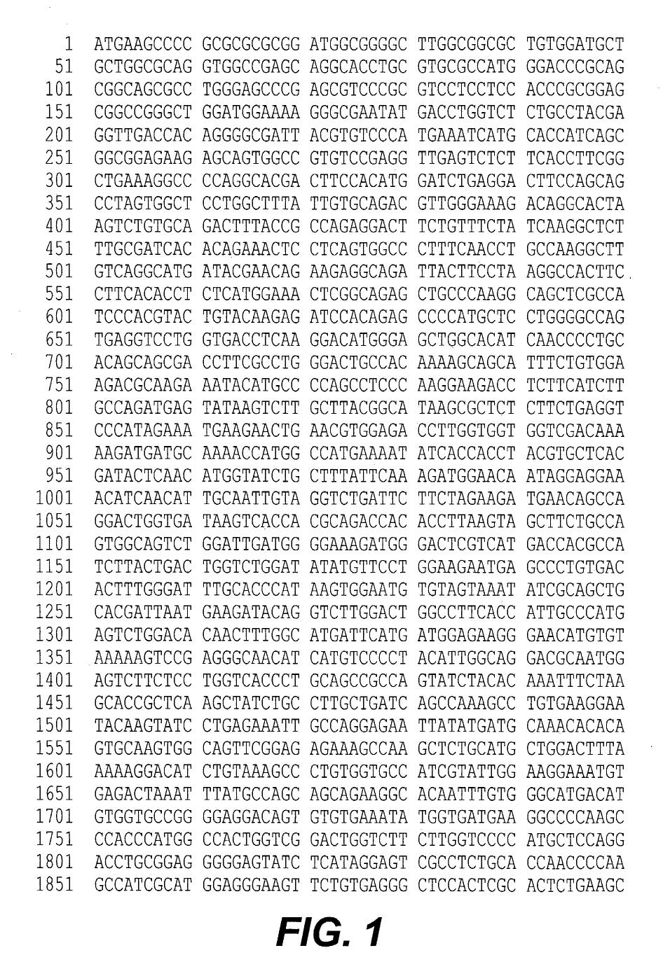

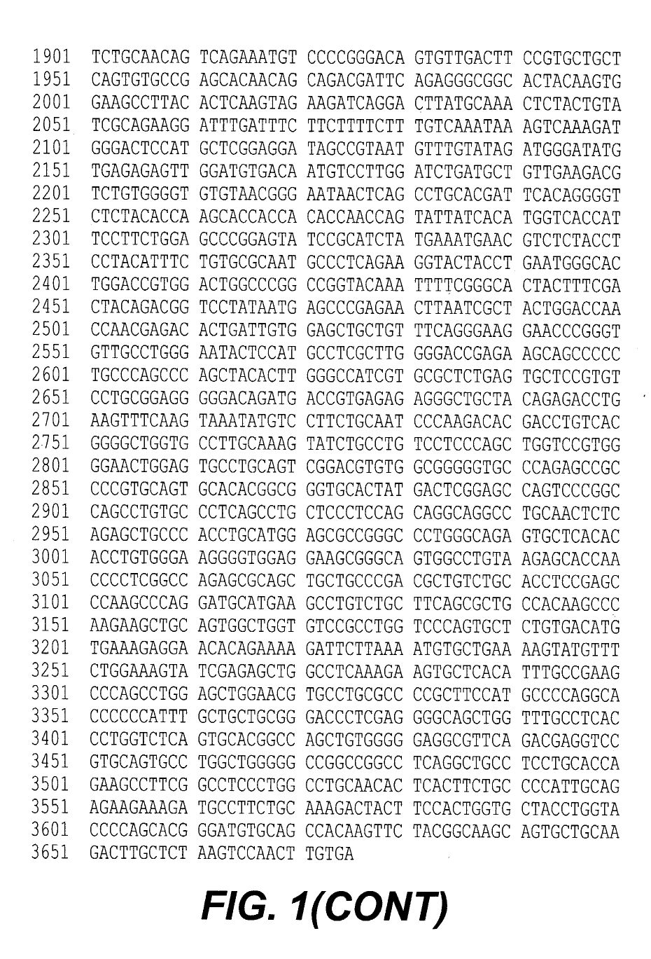

The gene for ADAMTS16 was isolated using a PCR strategy with tissue sources initially determined by preliminary PCR. Using 5P primer sequence 5P-GAGCACAACAGCAGACGATTCAG-3P (SEQ ID NO. 4) and 3P primer sequence 5P-GCGCACAGAAATGTAGGAGGTAGAGA-3P (SEQ ID NO. 5) on twelve different Marathon-Ready cDNAs from Clontech (Palo Alto, Calif., USA), a 408 base ‘Spacer domain’ fragment corresponding to nucleotide #1960 to 2367 of FIG. 1 was generated using the Advantage-GC2 PCR kit from Clontech. Reaction conditions were those recommended in the user manual and included 0.5 ng cDNA and 10 pmole of each primer per 50 μl reaction. Cycling conditions were as follows: one cycle of 94° C. for 1 min.; followed by 5 cycles consisting of 94° C. for 15 sec/72° C. for 3 min.; followed by 30 cycles consisting of 94° C. for 15 sec/68° C. for 3 min.; followed by one cycle of 68° C. for 3 min.

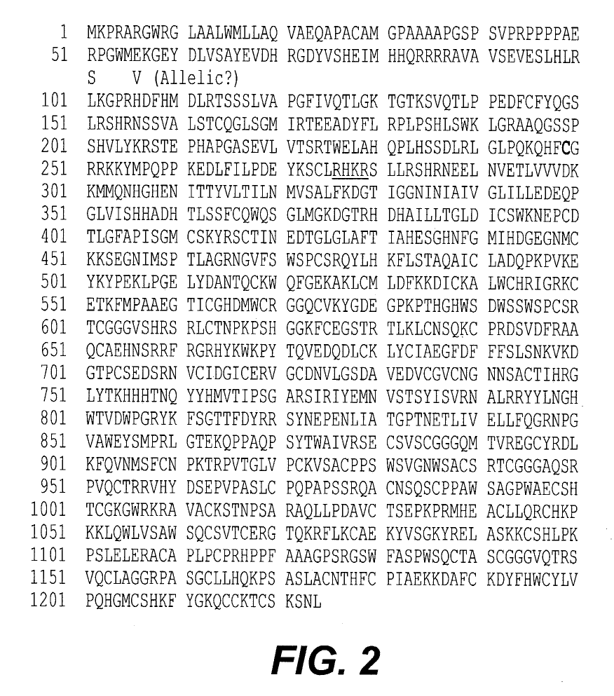

A 3704 base fragment of ADAMTS16 encompassing the predicted initiator Met through the stop codon was generated using PCR employing 5P primer sequence 5P-CGGAGCGCTCCTGGATGAA-3P (SEQ ID NO. 6) and 3P primer sequence 5P-GAGAGCGGTCCCAACTCACAAGT-3P (SEQ ID NO. 7) (extension of 14 bases upstream of the 5 prime end of sequence in FIG. 1 to 15 bases past the 3 prime end in FIG. 1). Human pancreas (pooled from 9 male/female Caucasians), uterus (pooled from ten Caucasians) or ovary (pooled from seven Caucasians) Marathon-Ready cDNA (Clontech) served as substrates and the MasterAmp High Fidelity Extra-Long PCR kit from Epicentre Technologies (Madison, Wis., USA) was used to perform the PCR reactions. Premix 4 was used as described in the user manual using 0.5 ng cDNA and 20 pmole of each primer per 50 μl reaction. Cycling conditions were as follows: 94° C. for 1 min., one cycle; followed by 35 cycles consisting of 94° C. for 20 sec/68° C. for 3 min.; followed by one cycle of 68° C. for 3 min. The PCR products resulting from these amplifications were ligated into the pPCR-ScriptAMP vector using the PCR-Script AMP Cloning Kit per manufacturer's instructions (Stratagene). Ligated products were transformed into ElectroMAX DH5α-E cells from Invitrogen (Carlsbad, Calif., USA). Clones originating from all libraries were sequenced to determine fidelity and consensus sequence.

Confirmation of the N-terminus of ADAMTS16 was achieved when a 1099 base fragment was generated from the uterus Marathon-Ready cDNA that overlapped the N-terminal region (nucleotide #1 to 461 of FIG. 1) of ADAMTS16 and extended 638 bases upstream to include an in frame stop codon. Primers (originating from the Celera Genscan predicted sequence) used to generate this fragment were 5P primer sequence 5P-CGCGGGCTGCAGGTGT-3P (SEQ ID NO. 8) and 3P primer sequence 5P-TGTGATCGCAAAGAGCCTTGAT-3P (SEQ ID NO. 9) (nucleotide #440 to 461, complement, FIG. 1). The MasterAmp High Fidelity Extra-Long PCR kit from Epicentre Technologies was used to perform the PCR reactions. Premix 4 was used as described in the user manual using 0.5 ng cDNA and 20 pmole of each primer per 50 μl reaction. Cycling conditions were as follows: 94° C. for 1 min., one cycle; followed by 35 cycles consisting of 94° C. for 20 sec/68° C. for 3 min.; followed by one cycle of 68° C. for 3 min. The PCR product resulting from this amplification was ligated into the pPCR-ScriptAMP vector using the PCR-Script AMP Cloning Kit per manufacturer's instructions (Stratagene, La Jolla, Calif., USA). Ligated products were transformed into ElectroMAX DH5α-E cells from Invitrogen. Several clones were sequenced and compared to determine a consensus sequence, to confirm the initiator methionine and to confirm a Genscan predicted upstream in frame stop codon.

Confirmation of the C-terminus of ADAMTS16was achieved when an 874 base fragment was generated from the uterus marathon cDNA that overlapped the final 581 nucleotides of the C-terminus of ADAMTS16 and extended an additional 293 bases. Primers (originating from the Celera Genscan predicted sequence) used to generate this fragment were 5P primer sequence 5P-CCGAGCCCAAGCCCAGGATGC-3P (SEQ ID NO. 10) (nucleotide #3095 to 3115, FIG. 1) and 3P primer sequence 5P-GAGTGCTGCCTCTCCCGTTGTGGTG-3P (SEQ ID NO. 11). The MasterAmp High Fidelity Extra-Long PCR kit from Epicentre Technologies was used to perform the PCR reactions. Premix 4 was used as described in the user manual using 0.5 ng cDNA and 20 pmole of each primer per 50 μl reaction. Cycling conditions were as follows: 94° C. for 1 min., one cycle; followed by 35 cycles consisting of 94° C. for 20 sec/68° C. for 3 min.; followed by one cycle of 68° C. for 3 min. The PCR product resulting from this amplification was ligated into the pPCR-ScriptAMP vector using the PCR-Script AMP Cloning Kit per manufacturer's instructions (Stratagene). Ligated products were transformed into ElectroMAX DH5α-E cells from Invitrogen. Several clones were sequenced and compared to determine a consensus sequence and confirmed the stop codon found at nucleotide #3673-3765 of FIG. 1.

A full determination of ADAMTS16 tissue distribution was achieved by probing a Clontech Human Multiple Tissue Expression Array 2 (MTE). A probe for the MTE was generated from a PCR product amplifying the spacer region of ADAMTS16 using 5P primer sequence 5P-GAGCACAACAGCAGACGATTCAG-3P (SEQ ID NO. 4) and 3P primer sequence 5P-GCGCACAGAAATGTAGGAGGTAGAGA-3P (SEQ ID NO. 5) (nucleotide #1960-2367 of FIG. 1) on human ovary Marathon-Ready cDNA. The MasterAmp High Fidelity Extra-Long PCR kit from Epicentre Technologies was used for the PCR reactions using premix 4 and standard conditions as described above. The PCR product resulting from this amplification was ligated into the pPCR-ScriptAMP vector using the PCR-Script AMP Cloning Kit per manufacturer's instructions (Stratagene). Ligated products were transformed into ElectroMAX DH5α-E cells from Invitrogen and sequenced. A probe encoding the spacer domain was obtained after digestion of the plasmid containing the PCR product with the polylinker restriction endonucleases NotI and BamHI (NEB) using conditions recommended by New England Biolabs. The 426 bp fragment was isolated using a 5% nondenaturing polyacrylamide gel using standard molecular biology techniques found in Maniatis's Molecular Cloning A Laboratory Manual. The fragment was electroeluted out of the gel slice using Sample Concentration Cups from Isco (Little Blue Tank). The purified spacer domain probe was radiolabelled using the Ready-To-Go DNA Labeling Beads (dCTP) from Amersham Pharmacia Biotech (Piscataway, N.J., USA) per the manufacturer's instructions. The radiolabelled fragment was purified away from primers and unincorporated radionucleotides using a Nick column from Amersham Pharmacia Biotech per the manufacturer's instructions and then used to probe the MTE. Manufacturer's conditions for hybridization of the MTE using a radiolabelled cDNA probe were followed. ADAMTS16 was found to be expressed in the following tissues: lymph node, fetal kidney, right cerebellum, left cerebellum, ovary, kidney, pancreas, tumor of the nervous system, lung, and bladder. Weaker expression was found in the following tissues: rectum, colon, jejunum, ileum, uterus, amygdala, hippocampus, stomach and esophagus.

The full-length sequence for ADAMTS16 was subcloned into expression vector pTMED2 in the following manner. Two duplexes encoding a vector EcoRI site (GAATTC) at the 5P end, optimized Kozac sequence (GCCGCCACC) upstream of the initiator Met (ATG), to the ADAMTS16 N-terminal SacII site (CCGCGG) located between nucleotides #143 and 148 in FIG. 1, were synthesized in the following oligonucleotides: 5P-AATTCGCCGCCACCATGAAGCCCCGCGCGCGCGGATGGCGGGGCTTGGCGG CGCTGTGGATGCTGCTGGCGCAGGTGGCCGAGCAGGCACCTGCGTG-3P (SEQ ID NO. 12) and complementary oligo 5P-GTGCCTGCTCGGCCACCTGCGCCAGCAGCATCCACAGCGCCGCCAAGCCCCG CCATCCGCGCGCGCGGGGCTTCATGGTGGCGGCG-3P (SEQ ID NO. 13) and CGCCATGGGACCCGCAGCGGCAGCGCCTGGGAGCCCGAGCGTCCCGCGTCC TCCTCCACCCGC-3P (SEQ ID NO. 14) and complementary oligo 5P-GGGTGGAGGAGGACGCGGGACGCTCGGGCTCCCAGGCGCTGCCGCTGCGGG TCCCATGGCGCACGCAG-3P (SEQ ID NO. 15). To construct an error free ADAMTS16, the duplexes were joined with a SacII-AatII fragment (nucleotide #143 to 22002 in FIG. 1) and AatII-NotI (nucleotide #2196 to end of sequence in FIG. 1) of two isolates of ADAMTS16 in the pPCR-ScriptAMP vector and together cloned into EcoRI-Not1I sites located in the polylinker of pTMED2.

The nucleotide coding sequence for ADAMTS16 from initiator methionine to stop codon is set forth in SEQ ID NO. 1. The predicted peptide sequence is set forth in SEQ ID NO. 2. There appears to be three allelic changes that alternated as a cluster among the clones and were not tissue specific in the tissues examined (uterus, pancreas). Six clones and celera genomic data had the sequences at amino acid numbers 18L (CTG), 104P (CCC), and 110M(ATG). Then other clones had the following changes: 18L(TTG), 104S(TCC), and 110V(GTG).

### Example 2

### Expression of Aggrecanase

In order to produce murine, human or other mammalian aggrecanase-related proteins, the DNA encoding it is transferred into an appropriate expression vector and introduced into mammalian cells or other preferred eukaryotic or prokaryotic hosts including insect host cell culture systems by conventional genetic engineering techniques. Expression systems for biologically active recombinant human aggrecanase are contemplated to be stably transformed mammalian cells, insect, yeast or bacterial cells.

One skilled in the art can construct mammalian expression vectors by employing a sequence comprising SEQ ID NO. 1 or other DNA sequences encoding aggrecanase-related proteins or other modified sequences and known vectors, such as, for example, pCD (Okayama et al., Mol Cell Biol, 2:161-170 (1982)), pJL3, pJL4 (Gough et al., EMBO J, 4:645-653 (1985)) and pMT2 CXM.

The mammalian expression vector pMT2 CXM is a derivative of p91023(b) (Wong et al., Science 228:810-815 (1985)) differing from the latter in that it contains the ampicillin resistance gene in place of the tetracycline resistance gene and further contains a XhoI site for insertion of cDNA clones. The functional elements of pMT2 CXM have been described (Kaufman, Proc. Natl. Acad. Sci. USA 82:689-693 (1985)) and include the adenovirus VA genes, the SV40 origin of replication including the 72 bp enhancer, the adenovirus major late promoter including a 5′ splice site and the majority of the adenovirus tripartite leader sequence present on adenovirus late mRNAs, a 3′ splice acceptor site, a DHFR insert, the SV40 early polyadenylation site (SV40), and pBR322 sequences needed for propagation in E. coli.

Plasmid pMT2 CXM was obtained by EcoRI digestion of pMT2-VWF, which has been deposited with the American Type Culture Collection (ATCC), Rockville, Md. (USA) under accession number ATCC 67122. EcoRI digestion excises the cDNA insert present in pMT2-VWF, yielding pMT2 in linear form which can be ligated and used to transform E. coli HB 101 or DH-5 to ampicillin resistance. Plasmid pMT2 DNA can be prepared by conventional methods. pMT2 CXM is then constructed using loopout/in mutagenesis (Morinaga, et al., Biotechnology 84: 636 (1984)). This removes bases 1075 to 1145 relative to the Hind III site near the SV40 origin of replication and enhancer sequences of pMT2. In addition it inserts the following sequence: 5′ PO-CATGGGCAGCTCGAG-3′ (SEQ. ID NO. 16) at nucleotide 1145. This sequence contains the recognition site for the restriction endonuclease Xho I. A derivative of pMT2CXM, termed pMT23, contains recognition sites for the restriction endonucleases PstI, Eco RI, SalI and XhoI. Plasmid pMT2 CXM and pMT23 DNA may be prepared by conventional methods.

pEMC2β1 derived from pMT21 may also be suitable in practice of the invention. pMT21 was derived from pMT2 which is derived from pMT2-VWF. As described above EcoRI digestion excises the cDNA insert present in pMT-VWF, yielding pMT2 in linear form which can be ligated and used to transform E. Coli HB 101 or DH-5 to ampicillin resistance. Plasmid pMT2 DNA can be prepared by conventional methods.

pMT21 was derived from pMT2 through the following two modifications. First, 76 bp of the 5′ untranslated region of the DHFR cDNA including a stretch of 19 G residues from G/C tailing for cDNA cloning is deleted. In this process, a XhoI site was inserted to obtain the following sequence immediately upstream from DHFR:

(SEQ. ID NO. 17)

5′-CTGCAGGCGAGCCTGAATTCCTCGAGGCATCATG-3′
PstI Eco RI XhoI

Second, a unique ClaI site was introduced by digestion with EcoRV and XbaI, treatment with Klenow fragment of DNA polymerase I, and ligation to a ClaI linker (CATCGATG). This deletes a 250 bp segment from the adenovirus associated RNA (VAI) region but does not interfere with VAI RNA gene expression or function. pMT21 was digested with EcoRI and XhoI, and used to derive the vector pEMC2B1.

A portion of the EMCV leader was obtained from pMT2-ECAT1 (S. K. Jung, et al, J. Virol 63:1651-1660 (1989)) by digestion with Eco RI and PstI, resulting in a 2752 bp fragment. This fragment was digested with TaqI yielding an Eco RI-TaqI fragment of 508 bp which was purified by electrophoresis on low melting agarose gel. A 68 bp adapter and its complementary strand were synthesized with a 5′ TaqI protruding end and a 3′ XhoI protruding end which has the following sequence:

| | |
|---|---|
| 5′-CGAGGTTAAAAAACGTCTAGGCCCCCCGAACCACGGGGACGTGGTTTTCCTTTGAAAAACACGATTGC-3′ | (SEQ. ID NO. 18) |
| TaqI XhoI | |

This sequence matches the EMC virus leader sequence from nucleotide 763 to 827. It also changes the ATG at position 10 within the EMC virus leader to an ATT and was followed by a XhoI site. A three way ligation of the pMT21 Eco RI-XhoI fragment, the EMC virus EcoRI-TaqI fragment, and the 68 bp oligonucleotide adapter TaqI-XhoI adapter resulting in the vector pEMC2β1.

This vector contains the SV40 origin of replication and enhancer, the adenovirus major late promoter, a cDNA copy of the majority of the adenovirus tripartite leader sequence, a small hybrid intervening sequence, an SV40 polyadenylation signal and the adenovirus VA I gene, DHFR and β-lactamase markers and an EMC sequence, in appropriate relationships to direct the high level expression of the desired cDNA in mammalian cells.

The construction of vectors may involve modification of the aggrecanase-related DNA sequences. For instance, aggrecanase cDNA can be modified by removing the non-coding nucleotides on the 5′ and 3′ ends of the coding region. The deleted non-coding nucleotides may or may not be replaced by other sequences known to be beneficial for expression. These vectors are transformed into appropriate host cells for expression of aggrecanase-related proteins. Additionally, the sequence of SEQ ID NO. 1 or other sequences encoding aggrecanase-related proteins can be manipulated to express a mature aggrecanase-related protein by deleting aggrecanase encoding propeptide sequences and replacing them with sequences encoding the complete propeptides of other aggrecanase proteins.

One skilled in the art can manipulate the sequences of SEQ ID NO. 1 by eliminating or replacing the mammalian regulatory sequences flanking the coding sequence with bacterial sequences to create bacterial vectors for intracellular or extracellular expression by bacterial cells. For example, the coding sequences could be further manipulated (e.g., ligated to other known linkers or modified by deleting non-coding sequences therefrom or altering nucleotides therein by other known techniques). The modified aggrecanase-related coding sequence could then be inserted into a known bacterial vector using procedures such as described in Taniguchi et al., Proc Natl Acad Sci USA, 77:5230-5233 (1980). This exemplary bacterial vector could then be transformed into bacterial host cells and an aggrecanase-related protein expressed thereby. For a strategy for producing extracellular expression of aggrecanase-related proteins in bacterial cells, see, e.g., European patent application EPA 177,343.

Similar manipulations can be performed for the construction of an insect vector (see, e.g. procedures described in published European patent application EPA 155,476) for expression in insect cells. A yeast vector could also be constructed employing yeast regulatory sequences for intracellular or extracellular expression of the factors of the present invention by yeast cells. (See, e.g., procedures described in published PCT application WO86/00639 and European patent application EPA 123,289).

A method for producing high levels of a aggrecanase-related protein of the invention in mammalian, bacterial, yeast or insect host cell systems may involve the construction of cells containing multiple copies of the heterologous aggrecanase-related gene. The heterologous gene is linked to an amplifiable marker, e.g., the dihydrofolate reductase (DHFR) gene for which cells containing increased gene copies can be selected for propagation in increasing concentrations of methotrexate (MTX) according to the procedures of Kaufman and Sharp, J Mol Biol, 159:601-629 (1982). This approach can be employed with a number of different cell types.

For example, a plasmid containing a DNA sequence for an aggrecanase-related protein of the invention in operative association with other plasmid sequences enabling expression thereof and the DHFR expression plasmid pAdA26SV(A)3 (Kaufman and Sharp, Mol Cell Biol 2:1304 (1982)) can be co-introduced into DHFR-deficient CHO cells, DUKX-BII, by various methods including calcium phosphate coprecipitation and transfection, electroporation or protoplast fusion. DHFR expressing transformants are selected for growth in alpha media with dialyzed fetal calf serum, and subsequently selected for amplification by growth in increasing concentrations of MTX (e.g. sequential steps in 0.02, 0.2, 1.0 and 5 μM MTX) as described in Kaufman et al., Mol Cell Biol., 5:1750 (1983). Transformants are cloned, and biologically active aggrecanase expression is monitored by the assays described above. Aggrecanase protein expression should increase with increasing levels of MTX resistance. Aggrecanase proteins are characterized using standard techniques known in the art such as pulse labeling with ³⁵S methionine or cysteine and polyacrylamide gel electrophoresis. Similar procedures can be followed to produce other related aggrecanase-related proteins.

In one example the aggrecanase gene of the present invention set forth in SEQ ID NO. 1 may be cloned into the expression vector pED6 (Kaufman et al., Nucleic Acid Res 19:44885-4490 (1991)). COS and CHO DUKX B11 cells were transiently transfected with the aggrecanase sequence of the invention (+/−co-transfection of PACE on a separate pED6 plasmid) by lipofection (LF2000, Invitrogen). Duplicate transfections are performed for each gene of interest: (a) one for harvesting conditioned media for activity assay and (b) one for ³⁵S methionine/cysteine metabolic labeling.

On day one media was changed to DME(COS) or alpha(CHO) media+1% heat-inactivated fetal calf serum+/−100 μg/ml heparin on wells(a) to be harvested for activity assay. After 48 h (day 4), conditioned media was harvested for activity assay.

On day 3, the duplicate wells (b) were changed to MEM (methionine-free/cysteine free) media+1% heat-inactivated fetal calf serum+100 μg/ml heparin+100 μCi/ml 35S-methioine/cysteine (Redivue Pro mix, Amersham). Following 6 h incubation at 37° C., conditioned media was harvested and run on SDS-PAGE gels under reducing conditions. Proteins were visualized by autoradiography.

In another example, the aggrecanase gene of the present invention set forth in SEQ ID NO.: 1 may be cloned into expression vector pHTop, a derivative of pED (Kaufman et al, 1991 NAR 19:4485-4490) in which the majority of the adenomajor late promoter was replaced by six repeats of the tet operator (described in Gossen et al, 1992, PNAS, 89:5547-5551). This vector contains the dihydrofolate reductase gene and when introduced in the cell line CHO/A2 (see description below) functions very efficiently and high expressors can be selected by isolating cells surviving in high Methotrexate concentrations.

Establishment of CHO stable cell lines: The CHO/A2 cell line was derived from CHO DUKX B11 (Urlaub and Chasin, 1980, PNAS USA 77:4216-4220) by stably integrating a transcriptional activator (tTA), a fusion protein between the Tet repressor and the herpes virus VP16 transcriptional domain (Gossen et al). A CHO cell line expressing extracellular ADAMTS8 was established by transfecting (lipofection) pHTopADAMTS8-Streptavidin tagged DNA into CHO/A2 cells and selecting clones in 0.02, 0.05 and 0.01 μM Methotrexate.

Screening of CHO stable cell lines: Multiple clones were screened by Western Blot using a streptavidin HRP antibody. The best clone was determined by virtue of its high expression and was one which resulted from 0.02 μM MTX selection and was chosen to be scaled up for roller bottle conditioned media production (4 Liters). The cell line was sent for large scale production.

### Example 3

### Biological Activity of Expressed Aggrecanase

To measure the biological activity of the expressed aggrecanase-related proteins obtained in Example 2 above, the proteins are recovered from the cell culture and purified by isolating the aggrecanase-related proteins from other proteinaceous materials with which they are co-produced as well as from other contaminants. Purification is carried out using standard techniques known to those skilled in the art. The purified protein may be assayed in accordance with the following assays:

Assays specifically to determine if the protein is an enzyme capable of cleaving aggrecan at the aggrecanase cleavage site:

1. Flourescent peptide assay: Expressed protein is incubated with a synthetic peptide which encompasses amino acids at the aggrecanase cleavage site of aggrecan. One side of the synthetic peptide has a flourophore and the other a quencher. Cleavage of the peptide separates the flourophore and quencher and elicits flourescence. From this assay it can be determined that the expressed protein can cleave aggrecan at the aggrecanase site, and relative flourescence tells the relative activity of the expressed protein.

2. Neoepitope western: Expressed protein is incubated with intact aggrecan. After several biochemical manipulations of the resulting sample (dialysis, chondroitinase treatment, lyophilization and reconstitution) the sample is run on an SDS PAGE gel. The gel is incubated with an antibody that only recognizes a site on aggrecan exposed after aggrecanase cleavage. The gel is transferred to nitrocellulose and developed with a secondary antibody (called a western assay) to result in bands running at a molecular weight consistent with aggrecanase generated cleavage products of aggrecan. This assay tells the expressed protein cleaved native aggrecan at the aggrecanase cleavage site, and also tells the molecular weight of the cleavage products. Relative density of the bands can give some idea of relative aggrecanase activity.

Assay to determine if an expressed protein can cleave aggrecan anywhere in the protein (not specific to the aggrecanase site):

3. Aggrecan ELISA: Expressed protein is incubated with intact aggrecan which had been previously adhered to plastic wells. The wells are washed and then incubated with an antibody that detects aggrecan. The wells are developed with a secondary antibody. If there is the original amount of aggrecan remaining in the well, the antibody will densely stain the well. If aggrecan was digested off the plate by the expressed protein, the antibody will demonstrate reduced staining due to reduced aggrecan concentration. This assay tells whether an expressed protein is capable of cleaving aggrecan (anywhere in the protein, not only at the aggrecanase site) and can determine relative aggrecan cleaving.

Protein analysis of the purified proteins is conducted using standard techniques such as SDS-PAGE acrylamide (Laemmli, Nature 227:680 (1970)) stained with silver (Oakley, et al., Anal Biochem. 105:361 (1980)) and by immunoblot (Towbin, et al., Proc. Natl. Acad. Sci. USA 76:4350 (1979)). Using the above described assays, expressed aggrecanase-related proteins are evaluated for their activity and useful aggrecanase-related molecules are identified.

### Example 4

### Preparation of Antibodies

An antibody against a novel aggrecanase molecule is prepared. To develop an antibody capable of inhibiting aggrecanase activity, a group of mice are immunized every two weeks with a novel aggrecanase protein mixed in Freunds complete adjuvant for the first two immunizations, and incomplete Freunds adjuvant thereafter. Throughout the immunization period, blood is sampled and tested for the presence of circulating antibodies. At week 9, an animal with circulating antibodies is selected, immunized for three consecutive days, and sacrificed. The spleen is removed and homogenized into cells. The spleen cells are fused to a myeloma fusion partner (line P3-x63-Ag8.653) using 50% PEG 1500 by an established procedure (Oi & Herzenberg, Selected Methods in Cellular Immunology, W. J. Freeman Co., San Francisco, Calif., at 351 (1980)). The fused cells are plated into 96-well microtiter plates at a density of 2×10⁵ cells/well. After 24 hours, the cells are subjected to HAT selection (Littlefield, Science, 145: 709 (1964)) effectively killing any unfused and unproductively fused myeloma cells.

Successfully fused hybridoma cells secreting anti-aggrecanase antibodies are identified by solid and solution phase ELISAs. Novel aggrecanase protein is prepared from CHO cells as described above and coated on polystyrene (for solid phase assays) or biotinylated (for a solution based assay). Neutralizing assays are also employed where aggrecan is coated on a polystyrene plate and biotin aggrecanase activity is inhibited by the addition of hybridoma supernatant. Results identify hybridomas expressing aggrecanase antibodies. These positive clones are cultured and expanded for further study. These cultures remain stable when expanded and cell lines are cloned by limiting dilution and cryopreserved.

From these cell cultures, a panel of antibodies is developed that specifically recognize aggrecanase proteins. Isotype of the antibodies is determined using a mouse immunoglobulin isotyping kit (Zymed™ Laboratories, Inc., San Francisco, Calif.).

### Example 5

### Method of Detecting Level of Aggrecanase

The anti-aggrecanase antibody prepared according to Example 4 can be used to detect the level of aggrecanase in a sample. The antibody can be used in an ELISA, for example, to identify the presence or absence, or quantify the amount of, aggrecanase in a sample. The antibody is labeled with a fluorescent tag. In general, the level of aggrecanase in a sample can be determined using any of the assays disclosed in Example 3.

### Example 6

### Method of Treating a Patient

The antibody developed according to Example 4 can be administered to patients suffering from a disease or disorder related to the loss of aggrecan, or excess aggrecanase activity. Patients take the composition one time or at intervals, such as once daily, and the symptoms and signs of their disease or disorder improve. For example, loss of aggrecan would decrease or cease and degradation of articular cartilage would decrease or cease. Symptoms of osteoarthritis would be reduced or eliminated. This shows that the composition of the invention is useful for the treatment of diseases or disorders related to the loss of aggrecan, or excess aggrecanase activity. The antibodies can also be used with patients susceptible to osteoarthritis, such as those who have a family history or markers of the disease, but have not yet begun to suffer its effects.

| Patient's | Route of | | | Predicted |
|---|---|---|---|---|
| Condition | Administration | Dosage | Frequency | Results |
| Osteoarthritis | Subcutaneous | 500 | Daily | Decrease in |
| | | μg/kg | | symptoms |
| ″ | ″ | 1 | Weekly | Decrease in |
| | | mg/kg | | symptoms |
| ″ | Intramuscular | 500 | Daily | Decrease in |
| | | μg/kg | | symptoms |
| ″ | ″ | 1 | Weekly | Decrease in |
| | | mg/kg | | symptoms |
| ″ | Intravenous | 500 | Daily | Decrease in |
| | | μg/kg | | symptoms |
| ″ | ″ | 1 | Weekly | Decrease in |
| | | mg/kg | | symptoms |
| Family History of | Subcutaneous | 500 | Daily | Prevention of |
| Osteoarthritis | | μg/kg | | condition |
| Family History of | Intramuscular | 500 | Daily | Prevention of |
| Osteoarthritis | | μg/kg | | condition |
| Family History of | Intravenous | 500 | Daily | Prevention of |
| Osteoarthritis | | μg/kg | | condition |

The foregoing descriptions detail presently preferred embodiments of the present invention. Numerous modifications and variations in practice thereof are expected to occur to those skilled in the art upon consideration of these descriptions. Those modifications and variations are believed to be encompassed within the claims appended hereto. All of the documents cited in this application are incorporated by reference in their entirety. Additionally, all sequences cited in databases and all references disclosed are incorporated by reference in their entirety.

What is claimed is:

1. An isolated DNA molecule comprising a DNA sequence chosen from:
a) the sequence of SEQ ID NO. 1 from nucleotide #1-#3675;
b) fragments of SEQ ID NO. 1;
c) homologous sequences of SEQ ID NO. 1;
d) sequences which hybridize under stringent conditions with SEQ ID NO. 1; and
e) naturally occurring human allelic sequences and equivalent degenerative codon sequences of (a) to (d).

2. A vector comprising a DNA molecule of claim 1 in operative association with an expression control sequence therefor.

3. A host cell transformed with the DNA sequence of claim 1.

4. A host cell transformed with a DNA sequence of claim 2.

5. A method for producing a purified human aggrecanase protein, said method comprising:
a) culturing a host cell transformed with a DNA molecule according to claim 1; and
b) recovering and purifying said aggrecanase protein from the culture medium.

6. The method of claim 5, wherein said host cell is an insect cell.

7. A purified aggrecanase protein comprising an amino acid sequence chosen from:
a) the amino acid sequence of SEQ ID NO. 2 from amino acid #1-#1224;
b) fragments of SEQ ID NO. 2; and
c) homologous aggrecanase proteins consisting of addition, substitution, and deletion mutants of the sequences of (a) to (b).

8. A purified aggrecanase protein produced by the steps of
a) culturing a cell transformed with a DNA molecule according to claim 1; and
b) recovering and purifying from said culture medium a protein comprising an amino acid sequence chosen from SEQ. ID NO. 2.

9. An antibody that binds to a purified aggrecanase protein of claim 7.

10. The antibody of claim 9, wherein the antibody inhibits aggrecanase activity.

11. A method for identifying inhibitors of aggrecanase comprising
a) providing an aggrecanase protein chosen from SEQ ID NO. 2 or a fragment thereof;
b) combining the aggrecanase with a potential inhibitor; and
c) evaluating whether the potential inhibitor inhibits aggrecanase activity.

12. The method of claim 11 wherein the method comprises evaluating the aggrecanase protein is used in a three dimensional structural analysis prior to combining with the potential inhibitor.

13. The method of claim 11 wherein the method comprises evaluating the aggrecanase protein is used in a computer aided drug design prior to combining with the potential inhibitor.

14. A pharmaceutical composition for inhibiting the proteolytic activity of aggrecanase, wherein the composition comprises an antibody according to claim 9 and a pharmaceutical carrier.

15. A method for inhibiting aggrecanase in a mammal comprising administering to said mammal an effective amount of the composition of claim 14 and allowing the composition to inhibit aggrecanase activity.

16. The method of claim 15, wherein the composition is administered intravenously, subcutaneously, or intramuscularly.

17. The method of claim 15, wherein the composition is administered at a dosage of from 500 μg/kg to 1 mg/kg.
